(19) United States
(12) Patent Application Publication
(10) Pub. No.: US 2006/0121468 A1
(43) Pub. Date: Jun. 8, 2006

(54) Viruses and virus-like particles for multiple antigen and target display

(76) Inventors: F. C. Thomas Allnutt, Glenwood, MD (US); David J. Kyle, Catonsville, MD (US)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 10/519,114

(22) PCT Filed: Jun. 25, 2003
(86) PCT No.: PCT/US03/19855

§ 371 Date: Nov. 14, 2005

Related U.S. Application Data
(60) Provisional application No. 60/391,429, filed on Jun. 26, 2002.

(51) Int. Cl. C12Q 1/70; C12Q 1/68

(52) U.S. Cl. 435/6; 435/5

(57) ABSTRACT

The present invention relates to the display of antigenic or allergenic components along with a tissue-targeting component on viruses or virus-like particles. Capsid protein genes are recombinantly modified to contain the specified components then expressed within a host organism, such as yeasts, bacteria, or algae, and allowed to spontaneously form active virus particles or virus-like particles. The recombinant complexes (virus or virus-like particle) can then be purified or used in situ as a therapeutic tool for disease or allergy prevention. The expression of multivalent and multifunctional components to increase the immunogenicity of the recombinant complexes, especially on oral administration, is provided.

### CROSS REFERENCE TO RELATED APPLICATION

This application is related to provisional application 60/391,429, filed in the United States Patent and Trademark Office on Jun. 26, 2002, the disclosure of which is herein incorporated by reference.

### BACKGROUND OF THE INVENTION

1. Technical Field

This invention is directed to methods for molecular manipulation of viruses and virus-like particles to produce therapeutically active materials useful for disease and allergy prevention.

2. Background Art

Viruses and virus-like particles (VLPs) have been manipulated for potential use as therapeutic compounds (Chapman et al., 2001; Hunt, 2002; Lomonossoff and Johnson, 1992; Porta et al., 1996). The use of VLPs to express multiple foreign epitopes is not new. The VLP format has previously been used to express three different epitopes on VLPs in insect cell culture (Buonaguro et al., 2001). This multiple display of the various HIV epitopes did not affect the Pr55 (gag) VLP formation. Epitopes expressed using phage displayed Hepatitis B virus epitope S (28-39) provided a hepatitis B virus-specific cytotoxic T lymphocyte response after injection of the phage particles without adjuvant into BALB/c mice (Wan et al., 2001). Therefore, it is possible to express various disease specific antigens and targeting moieties on the same VLP.

The use of edible vaccines (e.g., potatoes, tomatoes, and bananas) provides a simpler approach to vaccine development than the use of purified injectable vaccines (Tacket et al., 1998; Tacket et al., 2000). Oral vaccines bypass the requirement for pharmaceutical clean rooms, fermentors and extraction facilities, and rely instead on biological production methods based on current food and modern molecular biology. Virus-like particle (VLP) or pseudovirus-based approaches, using bacterial or yeast expression of capsid proteins that self-assemble into a non-virulent virus-like particle, stimulate both mucosal and cytotoxic T-lymphocyte (CTL) responses (Shi et al., 2001).

There are many literature sources that establish that an immunological response will occur via either mucosal or humoral (CTL) systems if an antigen can be presented to the intestinal mucosa (Gomez-Duarte et al., 2001). For example, stimulation of serum anti-tetanus toxin fragment C (TTFC) antibody production is observed following ingestion of recombinant Lactobacillus lactis producing the TTFC (Robinson and al., 1997). Also, production of serum anti-pneumococcal surface protein A antibodies is enhanced after ingestion of attenuated Salmonella-expressing PspA (Nayak, 1998). Additionally, oral immunization provided resistance to Streptococcus pneumoniae challenges (Nayak, 1998).

Arntzen and colleagues stimulated production of anti-hepatitis B (HVB) antibodies after the ingestion of bananas genetically modified to express the HVB coat protein (Arntzen et al., 2001; Arntzen and Lam, 2000). Thus, if the correct antigen is expressed in yeast or other host cells, immunization can take place through ingestion. By consuming the host cell biomass (e.g., yeast), the antigen is delivered directly to the intestinal mucosa, where its sensitization will lead to an immunological response. Current technology is encumbered by the lack of a sufficiently aggressive host response (i.e., too much antigen is required for oral vaccination to become universal).

One problem with oral vaccines is the amount of antigen needed to produce a sufficient immune response (e.g., 10-20 μg injected with adjuvant versus 750 μg orally administered). Purified antigens tend to be rapidly broken down (i.e., digested) in the stomach, such that they lose the ability to stimulate mucosal immunity. Bioencapsulation in cells, such as in plant oral vaccines, improves the antigen presentation, but large amounts of antigen are still required. Binding of the antigen to virus-like particles was demonstrated to provide a 1000-fold enhancement of the immune response compared to naked antigen (Chackerian et al., 1999, 2001). In that study, a conjugate of TNF-alpha to the papillomavirus VLP provided an enhanced immunogenic reaction that was 1000-fold greater than the fusion protein alone. In addition, a fusion protein expressing the L1 protein of the papillomavirus was shown to provide immunoprotection against the papillomavirus (Yuan et al., 2001).

There are many known examples of VLPs in the prior art (Hu et al., 1999; Kapusta et al., 1999; Lehner et al., 1994; Lehner et al., 1993; Mason et al., 1996; Tacket et al., 2000; Yao and Vakharia, 1998). These particles have been investigated as oral and injected vaccines (Kapusta et al., 1999; Tacket et al., 2000). As of yet none are being used as vaccines on a commercial basis. The genes for viral coat proteins have been engineered, placed into expression systems (that are not their normal host), the proteins expressed, and virus-like particles made (Peason and Roy, 1993).

Attenuated live viruses have been used for vaccination against a number of human diseases (e.g., chickenpox and smallpox). Related viruses with different host preferences have also been used for vaccines in humans. However, despite the advances in these fields, the use of VLPs as an oral vaccine to both express disease antigens and targeting moieties has not been disclosed.

### DISCLOSURE OF INVENTION

The invention provides a genetically engineered, attenuated, non-human, specific virus that displays on the virus' exterior face at least one antigen and a tissue-targeting moiety that increase the potency of orally administered vaccines.

The invention improves the host immune response using the VLP display approach in three ways. First, the antigen can be presented on a solid phase (i.e., expressed as viral coat protein fusions). Second, the antigen can be encased in cells (bioencapsulated) for some measure of protection from degradation. Third, the antigen can be given an additional functionality that helps target the antigen to the mucosal surface and present the antigen to the immune system.

This invention's multifunctional VLP display provides improved antigenicity of the displayed protein/peptide, stabilizes the antigen for passage through the stomach, and targets the antigen to the intestinal lining for better exposure to the mucosal immune system.

The invention also provides a genetically engineered, attenuated, non-human, specific virus that displays on the virus' exterior face, at least one allergen and a tissue-targeting moiety that increases the potency of orally administered allergy treatments.

The invention further provides the display of antigenic or allergenic peptides on the inner coat proteins (as fusion protein(s)) of attenuated viruses in conjunction with the tissue-targeting moiety to enhance survival of the antigen.

The invention yet further provides methods of producing recombinant multivalent/multifunctional vaccines or allergy treatment products from recombinant attenuated virus.

The invention provides a genetically engineered construct expressing (1) virus coat proteins in non-host species that display antigenic peptides or proteins; and (2) a tissue-targeting moiety that will self-assemble into VLPs.

The invention also provides a genetically engineered construct expressing (1) virus coat proteins in non-host species that display allergenic peptides or proteins; and (2) a tissue-targeting moiety that will self-assemble into VLPs.

The invention contemplates alternative self-assembling viral systems that act in a manner analogous to that described above, to form multivalent/multifunctional particles.

The invention targets the antigen to the intestinal mucosa using a multivalent and multifunctional display, allowing the antigen to pause in its travels through the lumen and increases the likelihood of presentation to the immune system. The high levels of expression in yeast cells (or other host systems capable of high level protein expression), increased immunogenicity due to the solid phase display and tissue targeting to increase the likelihood of the antigen presenting to the immune system, as described by this invention, avoids the need for chronic or repeated exposures that could lead to tolerance of the presented antigens(s).

A number of different peptides and their respective receptors can be utilized as tissue targeting tools to place the multivalent/multifunctional VLP or recombinant virus near to the intestinal lining to more highly favor the presentation to the mucosal immune system or to the humoral immune system. The use of the protective antigen (or portions thereof) from the anthrax toxin is one possible such moiety that has a less characterized and specific action. However, there are many different gastric hormones or other intestinal receptor/ligand pairs that are of interest. The linear peptide cholecystokinin binds to the CCK_{A} and CCK_{B} receptors (located in the pancreatic acinar cells, brain and stomach). CCK exists as 8, 33, 38, and 59 amino acid variants, all fully functional. The CCK-8 (8 amino acid) version would be a suitable candidate for cloning into a construct as envisioned in this invention, providing a small footprint, and a targeting to the stomach wall. Other receptor ligands of interest, include, but are not limited to, gastrin, gastrin-14, minigastrin, pentagastrin, secretin, motilin, glicentin, glucagons, glucagon-like peptide 1, glucagon-like peptide 2, exedin-3, exendin-4, vasoactive intestinal peptide (VIP), gastric inhibitory peptide (GIP), transferrin, chemokines, TECK, thymus-expressed chemokine, lactin, prolactin, TGF-alpha, serotonin, lipid binding protein, fatty acid binding protein, parathyroid hormone, isatin, and ligands of the neuroendrocine vasoactive intestinal peptide receptors.

These and aspects of the invention are provided by one or more of the following embodiments. In one embodiment of the invention, the genetically modified virus will be used to infect a fish and express the recombinant coat protein(s) in the fish tissues. Because the virus is non-pathogenic to both fish and humans, a sustained infection in the fish will occur, and the fish will remain healthy through the infection. These fish will contain recombinant fusion proteins, probably in proportion to the m.o.i. of the recombinant virus. After harvest of infected fish, the tissues can be prepared in a manner that does not destroy the antigenic or allergenic activity of the insert, e.g., Japanese style raw fish, i.e., sushi or sashimi. The oral stimulation of the immune system can be used either to increase expression of antibodies (in the case of an oral vaccine) or to acclimate a patient to an allergen by chronic and controlled exposure to the allergen.

In one embodiment, this invention will produce a recombinant rVLP-based oral vaccine in yeast, based on the fish birnavirus IPNV genome. This method provides a relatively quick production process for an oral vaccine. Yeast VLPs containing protein inserts stimulate humoral and cell mediated immunity (Welsh et al., 1999). VLPs have been hypothesized to be useful for delivery of foreign epitopes from heterologous pathogens or of drugs that require delivery to the gastrointestinal tract (Estes and Morris, 1999). This invention will solve a number of problems commonly associated with oral vaccines in a system analogous to bacteria phage display.

### BRIEF DESCRIPTION OF THE DRAWING

FIG. 1. The figure provides a diagram of the VLP display technology as a schematic representation of how the invention is applied to an orally administered vaccine. A yeast cell is transformed by standard molecular methods with a molecular biological genetic construct or constructs that allow the expression of chimeric virus capsid proteins, expressed in the yeast system, to display a targeting moiety (P) and at least one disease antigen (At). These fusion proteins self-assemble into virus-like particles with the foreign epitopes displayed on the surface. The targeting moiety (P) helps deliver the antigen (At) to the cell surface (e.g., intestinal mucosa), to enhance stimulation of the immune response.

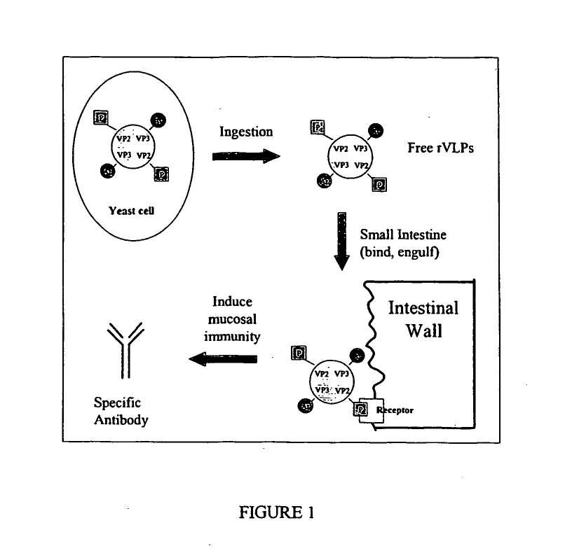

### MODES FOR CARRYING OUT THE INVENTION AND INDUSTRIAL APPLICABILITY

As used herein, a “virus-like particle” is a complex formed spontaneously from subunit proteins or peptides (e.g., by viral coat proteins) when placed together either in vivo or in vitro. For example, such a complex can be formed with a full complement of a specific virus' coat proteins or sub-sets of homologous or heterologous viral coat proteins, so long as they form complexes larger than single coat proteins. Any spontaneously formed complex from multimeric subunits can function under this definition (e.g., the plant phycobiliproteins can form spontaneously from subunits and used as a display system under this invention).

As used herein, a “target animal” is the organism in which the recombinant protein complex of this invention will have useful function. That is, once the specific displayed antigen or allergen is produced, it will be given (orally, by injection or other route) to a target animal to either induce an immunological response or affect the target animal for allergy treatment. Suitable target animals include human and non-human animals.

As used herein a “host organism” is the organism in which the virus or construct will multiply. This can either be an animal (e.g., fish or crustaceans), bacteria (e.g., E. coli, Bacillus sp., Bacillus subtilis, Streptomyces sp., Streptomyces griseus), algae (e.g., Phaeodactylum ticornutum, Chlorella sp., Chlorella vulgaris, Chlamydomonas reinhardtii, Synechocystis sp., Synechococcus sp., Spirulina sp., Spirulina platensis, Arthrospira sp.), and/or yeast (e.g., Pichia pastors, Saccharomyces cerevisiae, Hansenula sp.).

The term “exogenous sequence” refers to nucleic acid, usually DNA, inserted into an organism or virus in which it is not naturally present. Such sequence can be derived from a sequenced or synthetically produced strand or unknown DNA by insertion using ligation or other means.

### EXAMPLE 1

### Recombinant IPNV Engineered to Have Attenuated Virulence But to Express Specific HIV-1 Epitopes for Use as an Oral Vaccine

The infectious pancreatic necrosis virus (IPNV) is made synthetically by methods described by Vakharia et al. (Yao and Vakharia, 1998). The cloned virus can be manipulated by normal methods (Sambrook et al., 1989) to change the genes to encode regions of a proteinaceous antigen as a fusion with a capsid protein, which has been done previously for virus-like particles (Welsh et al., 1999). In this case, the HIV-1 epitopes (Buonaguro et al., 2001) are cloned onto the orf (open reading frame) for IPNV structural proteins VP2 and VP3 in a position that, when expressed and properly processed, forms a fusion protein of one of the capsid proteins that does not prevent assembly of the capsid.

The recombinant virus, prepared as described in Vakharia and Yao, 2001, but containing HIV-1 antigen(s), is used to infect fish in a fish farm (e.g., brook trout). The fish are then allowed to grow and, since the virus is non-pathogenic, can be harvested at a time when maximal antigen is detected in a sacrificed sampling of fish. The harvested fish are then made into a processed form that can be eaten in a manner that allows the antigen to function properly. Raw fish, such as Japanese style sushi or sashimi, provide a convenient method of transfer of this antigen as an oral vaccine. The sushi can be eaten to provide antigen and stimulate mucosal immunity to HIV-1. This is particularly important in HIV-1 since modes of infection are often associated with mucosal-tissues.

### EXAMPLE 2

### Recombinant Taura Syndrome Virus Expressing Antigenic Peptides as a Fusion with Viral Capsid Protein

Live recombinant viruses are made that contain antigenic peptide inserts in their capsid when assembled. An example is the use of Taura syndrome virus (TSV). This is a small virus that can be completely synthesized using existing molecular biological methods, e.g., PCR, from known sequence. A number of clones can be made that provide the entire genome and then specific sequence added that will allow a loop of antigenic material for a specific disease (e.g., parts of the hepatitis B surface antigen (Kong et al., 2001) to be exposed when the virus is allowed to infect its host.

In the example of rTSV, the virus is allowed to infect a tank containing shrimp such that the shrimp flesh will contain a large amount of the expressed virus. The shrimp flesh can then be used as an oral vaccine for hepatitis B. The use of live vaccines from non-human systems, such as the TSV, can provide a vector that does not replicate in humans. Humans that consume animals routinely consume viruses that infect the animals, e.g., humans consuming shrimp also consume viruses that infect shrimp, e.g., TSV, in the wild, or in aquacultural settings.

### EXAMPLE 3

### Purified Recombinant Attenuated Virus Used as a Vaccine Against HIV-1

HIV-1 epitope displayed on IPNV attenuated virus virions is isolated from the fish as infected from Example 1. The material is purified to homogeneity, tested for endotoxins, then aliquoted into sterile medium with or without adjuvant. This can then be utilized either as an oral or an injectable vaccine.

### EXAMPLE 4

### Use of Virus-Like Particles for Display of Proteins or Peptides Other than Those of the Parent Virus

VLPs are made up of virus capsid proteins that, when expressed in a viral infection or when expressed in a different expression system, are capable of self-assembly, either with DNA from the virus or as a pseudo-virus having only the assembled capsid (VLP). This can be with all of the capsid proteins or with some subset that will provide a suitable capsid like structure. In this invention, the capsid proteins will be known (sequence and possibly structure), such that genetic constructs can be made. The folded structure of the subunits can be predicted using existing software to model. Insertions of exogenous protein can be modeled to find regions that are predicted to not interfere with the proper folding and self-assembly of the VLP.

The use of a VLP as a vehicle for production of vaccines for diseases or conditions other than those caused by the virus used to produce the VLP is believed to be novel. This invention envisions a genetic construct made with one or many virus coat proteins that have been engineered to include portions of antigenic proteins from the disease target of interest, excluding those caused by the parent virus itself. Such a peptide insert(s) would have to allow free assembly of the viral coat but expose the antigenic peptide for the disease state on the surface of the VLP. Alternatively, the antigenic peptide can be expressed in a protected region of the capsid (e.g., inner protein coat) such that, on enzymatic degradation in the animal, the antigen is exposed, thus providing protection to the antigenic peptide as it passes through the digestive tract. Such a recombinant VLP (rVLP) would be used either purified or in the expression organism for vaccination against the disease target, either orally or by any other means.

Constructs containing exogenous DNA fused to the capsid protein(s) in such a manner to allow proper folding are made using the above concept. The capsid protein can contain a single peptide type or multiple peptide types fused in succession. If the VLP is made up of a single protein, it is likely that only a single peptide will be added. The rVLP will be expressed in some genetic system (e.g., yeast) such that the proteins will be expressed and assemble as VLPs that have an exogenous protein/peptide exposed externally. Such a peptide can be selected from a list, such as Fv fragments, antigenic fragments, binding sites, catalytic sites, biotin binding sites, streptavidin binding sites, or any other bioactive peptide. Such a rVLP can be isolated and used as a purified product or left in the expression system and fed as a complex feed containing this immunostimulating compound or bioactive compound.

### EXAMPLE 5

### Use of Virus-Like Particles for Expression of Multiple Antigenic or Allergenic Determinants

A recombinant virus like particle is made which expresses multiple determinants. One determinant targets the assembled particle in the host to a particular surface in the intestinal tract, allowing more rapid exposure of the antigenic portion (either by being on the correct surface, e.g., the stomach lining or upper intestine villi, or by being engulfed by endocytosis or other mechanisms). The other determinant(s) provide immunostimulating antigens for the disease to which the vaccine is targeted (e.g., HIV).

### EXAMPLE 6

### Non-Human Virus (Taura Syndrome Virus) Used as Display Medium

This invention also provides for the use of a functional/infective recombinant virus to accomplish immunostimulation analogous to the VLP example described above. The recombinant virus is non-human, and one that can be produced in bulk at a reasonable price. It can be a virus that infects yeast, bacteria, algae, plants, and/or animals. One approach is similar to that described in prior ABN patent applications, incorporated by reference, for vaccines expressed in crustaceans (60/318,867; U.S. Ser. No. 02/29081; U.S. provisional application of Barratt et al., filed May 29, 2003, application number pending). Here, a virus such as Taura syndrome virus (TSV) is constructed with nucleic acid inserted in areas where expression provides antigenic parts on the surface of the viral coat. The recombinant virus is used to infect a crustacean culture susceptible to the virus, and the flesh of the crustacean is harvested and used as an oral vaccine. A tissue targeting moiety can be linked to the antigen to allow it to be coexpressed and bind to a specific tissue.

### EXAMPLE 7

### Allergen Expression and Targeting

This invention also provides for the expression of protein or peptide allergens as fusion proteins with viral capsid proteins, as in Example 6, except that the expressed peptide is a common human allergen. It is expressed on one capsid protein of a virus, and a tissue-targeting moiety expressed on another capsid protein. A multivalent virus is formed, as in Example 6, and used for infection of either its host or cultured cells to provide enough whole virus for use in protection of the target animal by exposure therapy (gradual exposure to allergen via oral administration).

### EXAMPLE 8

### Double-Shelled Virus as Source of Inner and Outer Shell Based Virus-Like Particles for Display of Foreign Proteins or Peptides

Proteins for display can be placed either in the inner or outer shell of double shelled virus-like particles (e.g., Reoviridae or fijiviruses). The inner shell can be the allergen or the immunoantigen while the outer shell can act to protect the protein from gastric juices or digestion prior to entering the small intestine or lower intestine.

Using an inner viral coat protein construct containing an antigenic peptide insert as an immunostimulation system enhances antigen survival in the gut. A number of viruses have inner and outer capsids (Peason and Roy, 1993). In a viral genetic system, such as the bluetongue virus (BTV), one can envision that the heterologous antigenic protein is placed in a vector to allow expression as a fusion with inner capsid genes, then expressed with outer capsid proteins in order to produce a VLP that forms spontaneously. Such a rVLP can be introduced in purified or food form and possibly have enhanced survival through the digestive tract such that antigen is presented in a more complete form to tissues in the upper intestine.

### EXAMPLE 9

### Alternative Self-Assembling Viral Systems Used as Display Platforms

Also provided in this invention are alternative self-assembling viral systems that can act in a manner analogous to that described above. An example is the major coat protein of phage P22 and its scaffolding protein. Without the DNA, the scaffolding protein remains encased in the coat protein forming a double shelled viral ball (King et al., 1976). Such a system is useful in this invention.

### EXAMPLE 10

### Cholecystokinin Analog CCK-8 Used in Construct

Production of a construct using the 8 amino acid carboxyl terminus of cholecystokinin inserted into one of the capsid proteins provides a method for targeting the entire complex for binding to the stomach or intestinal cell wall at the CCK_{B} or CCK_{A} receptors.

### LITERATURE CITED

The following publications are relied upon and incorporated by reference herein.
Arntzen, C., H. S. Mason, and T. Haq. 2001. Oral immunization with transgenic plants. Texas A&M University System, UAS.
Arntzen, C. J., and D. M. Lam. 2000. Vaccines expressed in plants.
Buonaguro, L., F. M. Buonaguro, M. L. Tornesello, D. Mantas, E. Beth-Giraldo, R. Wagner, S. Michelson, M. C. Prevost, H. Wolf, and G. Giraldo. 2001. High efficient production of Pr55(gag) virus-like particles expressing multiple HIV-1 epitopes, including a gp120 protein derived from an Ugandan HIV-1 isolate of subtype A. Antiviral Res, 49:35-47.
Chackerian, B., D. R. Lowy, and J. T. Schiller. 1999. Induction of autoantibodies to mouse CCR5 with recombinant papillomavirus particles. Proc Natl Acad Sci USA, 96:2373-8.
Chackerian, B., D. R. Lowy, and J. T. Schiller 2001. Conjugation of a self-antigen to papillomavirus-like particles allows for efficient induction of protective autoantibodies. J Clin Invest, 108:415-23.
Chapman, S., S. Santa Cruz, K. Oparka, and T. Wilson. 2001. Method of producing a chimeric protein, U.S. Pat. No. 6,232,099.
Estes, M. K., and A. P. Morris. 1999. A viral enterotoxin. A new mechanism of virus-induced pathogenesis. Adv Exp Med Biol, 473:73-82.
Gomez-Duarte, O., M. Pasetti, A. Santiago, M. Sztein, S. Hoffman, and M. Levine. 2001. Expression, extracellular secretion, and immunogenicity of the Plasmodium falciparium sporozoite surface protein 2 in Salmonella vaccine strains. Infect Immun, 69:1192-1198.
Hu, Y. C., W. E. Bentley, G. H. Edwards, and V. N. Vakharia. 1999. Chimeric infectious bursal disease virus-like particles expressed in insect cells and purified by immobilized metal affinity chromatography. Biotechnol Bioeng, 63:721-9.
Hunt, N. 2002. Virus-like particles, their preparation and their use preferably in pharmaceutical screening and functional genomics, US Pub 20020052040 A 1.
Kapusta, J., A. Modelska, M. Figlerowicz, T. Pniewski, M. Letellier, O. Lisowa, V. Yusibov, H. Koprowski, A. Plucienniczak, and A. B. Legocki. 1999. A plant-derived edible vaccine against hepatitis B virus. Faseb J, 13:1796-9.
King, J., D. Botstein, S. Casjens, W. Earnshaw, S. Harrision, and D. Lenk. 1976. Structure and assembly of the capsid of bacteriophage P22. Philos Trans R Soc Lond B Biol Sci, 276:37-49.
Kong, Q., L. Richter, Y. F. Yang, C. J. Arntzen, H. S. Mason, and Y. Thanavala. 2001. Oral immunization with hepatitis B surface antigen expressed in transgenic plants. Proc Natl Acad Sci USA, 98:11539-44.
Kyle, D. J. 2001. Crustaceans as production systems for therapeutic proteins, U.S. Provisional Patent Application.
Lehner, T., L. A. Bergmeier, L. Tao, C. Panagiotidi, L. S. Klavinskis, L. Hussain, R. G. Ward, N. Meyers, S. E. Adams, A. J. Gearing, and et al., 1994. Targeted lymph node immunization with simian immunodeficiency virus p27 antigen to elicit genital, rectal, and urinary immune responses in nonhuman primates. J Immunol, 153:1858-68.
Lehner, T., R. Brookes, C. Panagiotidi, L. Tao, L. S. Klavinskis, J. Walker, P. Walker, R. Ward, L. Hussain, J. H. Gearing, and et al. 1993. T- and B-cell functions and epitope expression in nonhuman primates immunized with simian immunodeficiency virus antigen by the rectal route. Proc Natl Acad Sci USA, 90:8638-42.
Lomonossoff, G., and J. Johnson. 1992. Modified plant viruses as vectors, PCT WO 92/18618.
Mason, H. S., J. M. Ball, J. J. Shi, X. Jiang, M. K. Estes, and C. J. Arntzen. 1996. Expression of Norwalk virus capsid protein in transgenic tobacco and potato and its oral immunogenicity in mice. Proc Natl Acad Sci USA, 93:5335-40.
Nayak, A. 1998. A live recombinant avirulent oral Salmonella vaccine expressing pneumococcaccal surface protein A induced protective response against Streptococcus pneumoniae. Infect Immunol, 66:3744-3751.
Peason, L., and P. Roy. 1993. Genetically engineered multi-component virus-like particles as veterinary vaccines. Immunol Cell Biol, 71:381-389.
Porta, C., V. Spall, Lin T, J. Johnson, and G. Lomonossoff. 1996. The development of cowpea mosaic virus as a potential source of novel vaccines. Intervirology, 39:79-84.
Robinson, K., and e. al. 1997. Oral vaccination of mice against tetanus with recombinant Lactobacillus lactis. Nature Biotechnology, 15:653-657.
Sambrook, J., E. Fritsch, and T. Maniatis. 1989. Molecular Cloning: A Laboratory Manual. 2 ed. Cold Spring Harbor Press, Cold Spring Harbor.
Shi, W., J.-Y. Liu, Y. C. Huang, and L. Qiao. 2001. Papillomavirus pseudovirus: a novel vaccine to induce mucosal and systemic cytotoxic T-lymphocyte responses. J Virol, 75:10139-10148.
Tacket, C. O., H. S. Mason, G. Losonsky, J. D. Clements, M. M. Levine, and C. J. Arntzen. 1998. Immunogenicity in humans of a recombinant bacterial antigen delivered in a transgenic potato. Nat Med, 4:607-9.
Tacket, C. O., H. S. Mason, G. Losonsky, M. K. Estes, M. M. Levine, and C. J. Arntzen. 2000. Human immune responses to a novel norwalk virus vaccine delivered in transgenic potatoes. J Infect Dis, 182:302-5.
Vakharia, V., and K. Yao. 2001. Method for generating nonpathogenic infectious pancreatic necrosis virus (IPNV) from synthetic RNA transcripts, U.S. Pat. No. 6,274,147. Univ Md Biiotechnology Inst., USA.
Wan, Y., Y. Wu, J. Bian, X. Wang, W. Zhou, Z. Jia, Y. Tan, and L. Zhou. 2001. Induction of hepatitis B virus-specific cytotoxic T lymphocytes response in vivo by filamentous phage display vaccine. Vaccine, 19:2918-2923.
Welsh, M. D., D. R. Harper, M. Garcia-Valcarcel, W. J. Fowler, C. Aitken, D. J. Jeffries, and G. T. Layton. 1999. Ability of yeast Ty-VLPs (virus-like particles) containing varicella-zoster virus (VZV)gE and assembly protein fragments to induce in vitro proliferation of human lymphocytes from VZV immune patients. J Med Virol, 59:78-83.
Yao, K., and V. N. Vakharia. 1998. Generation of infectious pancreatic necrosis virus from cloned cDNA. J Virol, 72:8913-20.
Yuan, H., P. A. Estes, Y. Chen, J. Newsome, V. A. Olcese, R. L. Garcea, and R. Schlegel. 2001. Immunization with a pentameric L1 fusion protein protects against papillomavirus infection. J Virol, 75:7848-53.

We claim:

1. A method of producing a recombinant virus comprising:
(a) providing a viral genome;
(b) inserting one or more first exogenous sequences encoding a desired protein or peptide into the genome;
(c) inserting one or more second exogenous targeting sequences encoding a targeting element into the genome which has the function of targeting the complex to a specific location; and
(d) transfecting an appropriate host, and allowing the host to produce the virus.

2. The method of claim 1, wherein a first exogenous sequence encodes a protein or peptide that is antigenic for the target animal.

3. The method of claim 1, wherein a first exogenous sequence encodes a protein or peptide that is an allergen for the target animal.

4. The method of claims 1-3, wherein more than one first exogenous sequence is inserted.

5. A recombinant virus produced by the methods of claims 1-4.

6. A genetic construct comprising a viral genome with a first exogenous sequence for display of a peptide or protein on a viral capsid protein, and a second exogenous sequence for display of a targeting moiety.

7. The construct of claim 6, wherein the viral genome is modified to attenuate the virus in its natural host organism.

8. The construct of claim 6, wherein the exogenous sequences are inserted into a region or regions truncated to remove sequence unnecessary for viral replication.

9. The construct of claim 6, wherein the viral genome has been modified or truncated.

10. The construct of claim 6, wherein the first exogenous sequence is antigenic in an animal.

11. The construct of claim 6, wherein the first exogenous sequence is allergenic in an animal.

12. A recombinant virus produced from the genetic construct of claim 6.

13. A vaccine comprising a construct of claims 6-12.

14. A method of using the vaccine of claim 13, comprising:
(a) infecting an organism with a construct of claims 6-12; and
(b) orally feeding the whole biomass of the infected organism to human or non-human animals.

15. The method of claim 14, wherein the biomass has been processed for uniform dosing.

16. The method of claim 15, wherein the biomass is freeze dried.

17. The method of claim 15, wherein the biomass is encapsulated.

18. The vaccine of claim 13, wherein the vaccine is an oral vaccine.

19. The vaccine of claim 13, wherein the vaccine is an injectable vaccine.

20. A method of treating allergy in a subject in need of such treatment, comprising:
(a) providing the recombinant virus of claim 5 or claim 12; and
(b) administering the virus to the subject.

21. The method of claim 20, wherein the treatment is oral.

22. The method of claim 20, wherein the treatment is injectable.

23. The method of claims 20-21, further comprising:
(a) infecting an organism with the recombinant virus of claim 5; and
(b) orally feeding the whole biomass of the infected organism to human or non-human animals.

24. The method of claim 23, wherein the biomass has been processed for uniform dosing.

25. The method of claims 20-24, wherein the biomass is freeze dried.

26. The method of claims 20-24, wherein the biomass is encapsulated.

27. A method of producing a recombinant virus-like particle comprising:
(a) providing a viral genome;
(b) isolating at least one viral coat protein sequence;
(c) inserting at least one first exogenous sequence encoding a protein or peptide of interest into the coat protein sequences;
(d) inserting at least one second exogenous sequence encoding a targeting sequence;
(e) cloning the viral coat protein sequence comprising the first and second exogenous sequences into an appropriate vector; and
(f) transforming an appropriate host.

28. The method of claim 27, wherein the first exogenous sequence encodes a protein or peptide that is antigenic in an animal.

29. The method of claim 27, wherein the first exogenous sequence encodes a protein or peptide that is an allergen in an animal.

30. The method of claim 27, wherein more than one first exogenous sequences is inserted.

31. The method of claim 27, wherein one or more of the second exogenous sequences has the function of targeting the complex to a specific location.

32. The method of claim 27, wherein more than one viral coat protein is isolated.

33. A recombinant virus-like particle produced by the method of claims 27-32.

34. A genetic construct comprising at least one viral coat protein containing exogenous sequence for displayed peptides or proteins.

35. The construct of claim 34, wherein more than one viral coat protein has been modified to display foreign proteins or peptides.

36. The construct of claim 34, wherein more than one non-identical exogenous protein has been inserted.

37. The construct of claim 34, wherein the exogenous sequence is inserted into a region truncated to remove sequence unnecessary for virus-like particle self-assembly.

38. The genetic construct of claim 34, wherein the first exogenous sequence is antigenic in an animal.

39. The genetic construct of claim 34, wherein the first exogenous sequence is allergenic in an animal.

40. A recombinant virus-like particle produced from the genetic construct of claims 34-39.

41. A method of using the recombinant virus-like particle of claims 34-39 as a vaccine, comprising:
(a) providing the recombinant virus-like particle; and
(b) administering it to a subject.

42. The method of claim 41, further comprising:
(a) infecting an organism with the recombinant virus-like particle of claim 40; and
(b) orally feeding the whole biomass of the infected organism to human or non-human animals.

43. The method of claim 42, wherein the biomass is processed for uniform dosing.

44. The method of claims 41-43, wherein the biomass is freeze dried.

45. The method of claims 41-43, wherein the biomass is encapsulated.

46. The method of claims 41-46, wherein the vaccine is used as a treatment for allergy.

47. The method of claim 41, wherein the vaccine is administered by injection.

48. A vaccine comprising the recombinant virus-like particles of claims 34-39, wherein the particles are isolated.
